(19) United States
(12) Patent Application Publication
(10) Pub. No.: US 2002/0150925 A1
(43) Pub. Date: Oct. 17, 2002

(54) Biochip testing system

(76) Inventors: Guan-Ting Chen, Taipei (TW); Minda Lai, Jungli (TW)

Correspondence Address: MORRISON & FOERSTER LLP, 3811 VALLEY CENTRE DRIVE, SUITE 500, SAN DIEGO, CA, 92130-2332, US

(21) Appl. No.: 10/003,458

(22) Filed: Oct. 31, 2001

(30) Foreign Application Priority Data
Apr. 13, 2001 (TW) 90108957

(51) Int. Cl. C12Q 1/68; C12M 1/34

(52) U.S. Cl. 435/6; 435/287.2

(57) ABSTRACT

The invention relates to a biochip testing system for testing the status of a biochip carried on a recording medium. The biochip testing system comprises: a light transmitter; a biochip including a plurality of cells coated with biological reagents; a recording medium for carrying the biochip; and a light receiver for receiving the light from the light transmitter passing through or reflect from the biochip. The invention also relates to a recording medium having the biochip thereon, and a personal digital biochip assistant having the biochip testing system.

### BACKGROUND OF THE INVENTION

1. Field of the Invention

The invention relates to a biochip testing system for testing the status of a biochip carried on a recording medium. More specifically, the biochip testing system comprises: a light transmitter; a biochip having a plurality of cells each coated with a biological reagent; a recording medium for carrying the biochip; and a light receiver for receiving the light from the light transmitter passing through or reflect from the biochip and sensor. The invention also relates to a recording medium having the biochip thereon, and a personal digital biochip assistant having the biochip testing system.

2. Description of Prior Arts

Recently, biochips play an important role in gene engineering and against diseases. It's not only because of the variety of applications including diagnostics, drug screening, forensics, herb screening, and plant biology etc, but also because of the variety of sciences and technologies involved in the development process. Biochip development routinely draws on such diverse fields as biochemistry, nucleic acid chemistry, molecular biology, genetics, toxicology, material sciences, physical chemistry, electrical and mechanical engineering, optics, image and data analysis, database management, and automation (Cheung, V., M. Morley, F. Aguilar, A. Massimi, R. Kucherlapti, and G. Childs. 1999. Making and Reading Microarrays. Nat. Genet. 21:15-19). The promised dividends for investing in biochip development are increased information content, higher throughout, and smaller sample and reagent requirements. For the general biochips, coating biomolecules on a glass carrier, nylon carrier and so forth usually forms them. After forming the biochip, how to obtain the desired information from the biochip is another important issue.

The indication method applied in most of the current biochips is the well-known indication method for testing nucleic acids, such as DNA or RNA. Up to now, the indicating molecules frequently used for generating signals include isotope label particles, chemiluminescence elements, fluorescent dyes or non-fluorescent dyes. However, since the density of biochip is gradually increased, the pitch between the testing probes is narrowed, and the area of the biochip is reduced, a plurality of researches are focused upon developing an indication method having high signal strength and low background noise.

One of the keys to the automation of microarray technology is an integrated microarray scanning and analysis system. In most cases these scanners use lasers to illuminate one pixel at a time until all the spots on the gene chip have been scanned and recorded as a high-resolution image file. The scanned images are analyzed in a data extraction process that measurements the absolute and relative fluorescence at two wavelengths.

As mentioned above, two key factors to consider when evaluating a microarray detection system are the quality of the image and the time taken to generate such an image. In general terms, charge-coupled devices (CCDs) offer an advantage over photomultiplier tube (PMT) in that they allow simultaneous acquisition of relatively large image (approximately 1 cm2) (Schena, M. and R. W. Davis. 1999. Parallel Analysis with Biological Chips. PCR Applications, 445-456. Academic Press, San Diego.) The CCD systems typically use broadband xenon bulb technology and spectral filtration for excitation. Spectral filtration is a particularly important issue because most commonly used fluors have a small Stoke's shift (the difference in wavelength between the excitation and emission maxima of a fluorochrome), which makes the effective separation of excitation and emission light difficult.

Isotope label indication method though has the advantage of high signal strength, however, its operator requires special operating license and the waste derived therefrom is hard to process. Therefore, the cost of the isotope label indication method is high and thus it is not popular. The isotope label indication method is used in only the research rooms sponsored by national academy or university, and is not suitable for commercial use. In addition, because the radiative strength is high, it is easy to cause a phenomenon of interference between two testing points when the testing probe is closely to each other. Accordingly, the isotope label indication method is inappropriate to be used in high-density biochips.

Fluorescent indication method is the preferred indication method currently, because it may provide a desired signal strength without the problem of waste processing. However, the disadvantage of the fluorescent indication method is that it is apt to be interfered by the background noise, and thus a confocal laser scanner which may radiate high power is inevitably used to read signal generated therefrom. The confocal laser is very expensive so that is inappropriate to be commercially used. Moreover, when the confocal laser scanner is used as testing system to test the status of biochip, the interference caused by the background noise can be overcome because it may radiate high power. However, because it is required for the confocal laser scanner to use two or more laser heads together with a confocal system so as to form confocal to test biochip, the test path thereof is rather long, and the volume also rather large, which cause the increase of cost. In addition, after scanning the biochip by the confocal laser scanner, it is required to further carry out an image processing, and analyze the result of the image being processed. Therefore, in view of commercialization, the confocal laser scanner is improper for use in the testing of personalized biochip.

A laser-based detection system uses defined excitation wavelengths, which typically provides, in a cleaner excitation light, many more excitation photons delivered to the sample and many more emission photons generated and collected for each pixel in a give amount time. There are limitations to laser systems, for example, power and wavelength change as a function of temperature in both diode and solid-state lasers. To control for temperature effects, the lasers can be stabilized either for temperature or power. The lasers are temperature controlled, laser output at the source is continually monitored which photodiodes, and input current is automatically adjusted for any fluctuations detected.

In general, fabrication methods of DNA chip were put into three categories: photolithographic imprinting, ink-jet printing, and spotting technologies. In terms of the more complicated photolithographic imprinting method, nucleic acid probe is synthesized directly on the surface of carrier by photochemical synthesis and photolithographic mask techniques of semiconductor. Because of special and expensive scanners, peripherals, diagnostic kits, and analytic software being required in the process as well as the cost of photolithographic mask being so great, the method is still not a popular one. Non-contact, drop-on-demand, high-density ink-jet printing array also costs too much and has a low resolution. To solve such problems, we are going to develop an easy and high-resolution fabrication method suitable for mass production.

Optical fiber has been applied to transmittance of photoelectric image for years. Furthermore, refining of plastic fiber function has reduced the price of optical fiber. With its distinction, optical fiber can be utilized for microarray application. Because each fiber in optical fiber array can be treated as a cell, it is convenience to manufacture products. Selecting required fiber cell from gene bank to assemble an array could be an easy, flexible, timesaving, and suitable for customer-built production. Besides, image transmitted by fiber is so clear that sensitivity of diagnosis will be enhanced.

One of the Optical sensor array techniques developed in the prior art is based upon microspheres reacting with reactants and then randomly placing on etching template with fibers. To the contrary, our techniques are direct reaction by coated molecules on surface reacting with reactants to save process and materials. That means our techniques are ahead of others' in concept and practicability.

The Optical fiber array of the present invention and other gene chips could be complementary to each other. When a test does not need too many testing items or has to combine different kinds of testing items, mass production process will not work, but optical fiber array could. Besides, because of its uncomplicated process and better resolution, the cost of optical fiber array could be cut down. That gives the optical fiber array of the present invention an advantage over its opponents to capture a large market share.

Personal Digital Biochip Assistant (PDBA) is a device integrating biochip and storage disk into a card-sized apparatus. Such a chip disk could store patients'diagnostics information as a gene I.D to assist medical services.

In the prior art, DNA microarrays have revolutionized the collection and analysis of genetic information. The monitoring of RNA expression and DNA variations has contributed dramatically to our understanding of basic biology and is having a direct impact in the clinic. Most DNA microarrays are prepared with one of three now-standard approaches. The Affymetrix GeneChip probe arrays are prepared using patterned, light-directed combinatorial chemical synthesis (S. A. Fodor, Science 277, 393, 1997). Such arrays can contain hundreds to hundreds of thousands of probe sequences on a glass surface. To prepare spotted arrays, pins distribute preformed nucleic acid solutions to precise positions on various substrates. Arrays can also be created with ink-jet techniques in which oligonucleotides are synthesized base by base through sequential solution-based reactions on an appropriate substrate (A. P. Blanchard et al., Biosens. and Bioelectron. 11, 687, 1996). A relative newcomer to the array field is the self-assembled bead array. This format is a departure from these three approaches and offers the molecular biologist an entirely new platform on which to study gene expression and DNA variation.

A biochip detecting system is also known as being assembled on an optical fiber substrate. Optical fiber is made of two types of glass or plastic: the inner ring, the core, which has a slightly higher refractive index than the outer ring, is known as the cladding. Due to the mismatch in refractive indices, light is transmitted through the core over long distances by a process known as total internal reflection. The low-attenuation phenomenon is employed routinely to carry light signals encoding most of our high-speed communication systems with telephone, Internet, and video signals included.

Each of the optical fibers in the current invention, on the contrary, can be separately converted into DNA sensors by sticking a DNA probe to the distal tip or by removing the cladding and attaching the DNA probe to the outside of the core. Once hybridization to its fluorescent target, labeled double-stranded DNA is formed and be analyzed. At the time the light at an excitation wavelength is focused on the proximal end of the fiber, the fluorescent label on the distal terminal or on the core turns excited. Isotropically emitted light from the fluorophore is captured by the same fiber and sent back to the proximal terminal at which a detection system divides the excitation light signals from the emitted signals. By way of physically bundling multiple fibers together, simple DNA arrays can be made from such optical fibers, whose advantages are their small size and flexibility. Such features enable the sensors to be directly placed into sample solutions of DNA, instead of putting the samples onto the sensor's surface.

Images, formerly, could not be carried over conventional optical fibers, as the light signals became mixed and spatial resolution was not preserved. To image optical fibers created contains an array of thousands of densely packed individual optical fibers fused into a coherent unitary bundle (D. R. Walt, Acc. Chem. Res. 31, 267,1998). Typical imaging arrays contain between 5,000 and 50,000 individual fibers, each 3 to 7 μm in diameter, creating a entire array diameter of 300 to 1,000 μm. Each fiber has its own light signal; as a matter of course, such an array can be applied to build up images with a pixel-by-pixel image reconstruction similar to that of compound eyes of an insect. Moreover, images processing techniques can also be employed to evaluate the images and registered the positions of each cell type. Bead-based fiber-optic arrays (PCT application No. WO0071992) are distinguishedly different from other microarray formats where each probe in the array is not registered by deliberate positioning in array fabrication but spectrally registered following to its random distribution in the wells. Therefore, each array is unique with different microspheres positioned in different patterns among each array. There still are several disadvantages, thought bead-based fiber-optic arrays are rather simple to prepare. Owing to every array with the sensing probes arranged randomly, every array should be decoded. Each nucleotide probe still should be synthesized individually as opposed to the combinatorial synthesis approaches available with light-directed or ink-jet techniques.

In the present invention, the fibrous optic arrays can be prepared by attaching different probes to each fiber's distal surface. Individual populations of fibrous cells, each containing a different probe sequence, are then easy to form a stock. This library of fibrous cells can be combined different requirements to form fiber array. The fibrous cells are all the same size and are matched to the size of the distal surface so that any of the sequences can be positioned in any given cell and so a strategy must be devised for registering each array.

The present optic fibrous arrays, which can be directly applied to the sample particularly, facilitate the use of small sample sizes. Nucleic acids of sample sizes 1 μl could help to be detected following a limited number of amplification cycles. In the current invention optical fibers, especially, may be dipped directly into microtiter plates containing the target solution. As the sensors are in small size, diffusion limitations typical of large-area planar surfaces will be reduced. Moreover, a simple high-temperature denaturation or organic solvent treatment can achieve dehybridization. Furthermore, single-base mismatches between the probe and target could be detected if adjusting the stringency with either temperature or solvent.

The optical fibrous arrays of the present invention also provide a high degree of flexibility in diverse applications. As new probe fibrous cells are simply added to the probe library or new sequences of interest are identified. This flexibility and friendly especially offers an advantage to the individual researcher or user. Moreover, all the techniques for enhancing the flexibility of arrays for molecular and universal fabrication beacons for label-less detection can be used with the optical fibrous sensor array testing system.

The present application therefore is directed to a novel and improved biochip testing system, characterized in the application of optical fiber array technique, optionally, in combination with a demountable chip disk module. The present application is further directed to diagnostic chips for detecting specific diseases, and to personal digital biochip assistant adapted to lab-on-a-chip technology, by which improvements over the prior art are achieved, including the cost down in production and the flexibly to meet the requirements based on the customer's personal needs, in addition to the competence of recording patient's diagnosis history so as to keep tracking to the patient's case history.

### SUMMARY OF THE INVENTION

One object of the present invention is aimed at overcoming the problems existing in the prior art, by providing a biochip testing system, which permits the light to transmit through or reflect from the biochip with maximal power but without attenuation.

Another object of the invention is to provide a biochip testing system of which cost is reduced so that it is suitable for use in the test of personalized biochip.

The other object of the invention is to provide a biochip testing system, where the light, after passing through or reflect from a biochip, can be directly received by a light receiver, and the variation of light can be directly converted into corresponding data or image for analysis, such that the cost and processing time can be reduced, and the testing efficiency can be further improved.

To accomplish the above objects, according to a first aspect of the invention, there is provided a biochip testing system, comprising: a light transmitter; a biochip including a plurality of cells each coated with a biological reagent; a recording medium for carrying the biochip; and a light receiver for receiving the light from the light transmitter passing through or reflect from each cell of the biochip, thereby the status of each cell of the biochip may be tested through the variations of the light before and after passing through or reflect from the biochip.

In the biochip testing system of the first aspect of the invention, the biochip is either transparent or non-transparent. In the case that the biochip is transparent, the light transmitter and the light receiver are arranged on each sides of the biochip respectively. While in the case that the biochip is non-transparent, the light transmitter and the light receiver are disposed on the same side of the biochip.

In the biochip testing system of the first aspect of the invention, the light transmitter includes a light source, and the light source is preferably a laser light source, a LED light source, a LD light source, an UV light source, a haloid light source, or others.

In the biochip testing system of the first aspect of the invention, the biochip is formed by a polymer material carrier, such as glass carrier, nylon carrier, or optical fiber carrier, on which biological reagents are coated. Preferably, the optical fiber carrier is formed by gathering plural of plastic fibers, glass fibers, quartz fiber, and the like. Most preferred, the optical fiber is formed by gathering plural of plastic fibers. The core of plastic fiber is made of highly transparent polymers, mainly polymethylmetharylate (PMMA), and the coat of plastic fiber is primarily made of polyethylene. Standard outer diameter of plastic fiber could be 1000 μm, 750 μm, 500 μm, but outer diameter of plastic fiber used for microarray should be 75˜200 μm. PDBA is an application of embedded system, and embedded system integrates a lot of techniques, such as operation system, single chip, application software, readout device, storage system, fabrication of biochip, and sample labeling. Accordingly, PDBA should harmonize biotechnology, software information, chemical engineering, photoelectricity, and microelectronics micro-mechanics system (MEMS) to self fulfill.

In the biochip testing system of the first aspect of the invention, the biochip is formed by gathering plural of plastic fibers, glass fibers, or quartz fiber, etc., with one end of each fiber being coated with biological reagent, and surrounded by an opaque clading.

In the biochip testing system of the first aspect of the invention, the light receiver includes a photoelectric converter and a signal processing unit, where the photoelectric converter converted the light being received as an electronic signal, and the signal processing unit converted the electronic signal through current/voltage transform and analogue/digital transform as a digital signal and displayed on a display.

In the biochip testing system of the first aspect of the invention, the light receiver is the one selecting from the group consisting of CMOS sensors, CCD array, CCDs, photodiode array, photodiodes, and PMT.

In the biochip testing system of the first aspect of the invention, the display may be one selecting from the electronic devices consisting of personal computer, notebook, or portable electronic devices, such as personal digital assistant (PDA), palm-size PC, or smartphone, etc., and the electronic signal is transmitted to the electronic devices through an interface such as IEEE 1394, USB, or in wireless.

In the biochip testing system of the first aspect of the invention, the biochip is preferably detachably adhered on the recording medium.

In the biochip testing system of the first aspect of the invention, the light receiver is arranged in a single one or matrix, such that it may read the signal from the biochip, one-by-one or in array.

In the biochip testing system of the first aspect of the invention, the light receiver further comprises a writing head for writing the variation of signal sensored by the light receiver on the recording medium.

In the biochip testing system of the first aspect of the invention, the recording medium is one selecting from the group consisting of magnetic disc, optical disc, smart card, or others.

According to the second aspect of the invention, a personal digital biochip assistant (PDBA) comprising the biochip testing system of the first aspect is provided.

In the personal digital biochip assistant of the second aspect of the invention, the personal digital biochip assistant may present its analysis results on electronic devices through interfaces, or the PDBA is built all-in-one, that is, including the above said biochip testing system and display.

Additional features of the invention will be described hereinafter which form the subject of the claims of the invention. It should be appreciated by those skilled in the art that the conception and the disclosed specific embodiment may be readily utilized as a basis for modifying or designing other structures for carrying out the same purposes of the invention. It should also be realized by those skilled in the art that such equivalent constructions do not depart from the spirit and scope of the invention as set forth in the appended claims.

### BRIEF DESCRIPTION OF THE DRAWINGS

For a further understanding of the nature and objects of the invention, reference may be made to the following detailed description taken in conjunction with the accompanying drawings in which:

FIG. 1 is a block diagram of the biochip testing system according to the first embodiment of the invention, where the biochip 7 has been carried on the recording medium 3;

FIG. 2 is a block diagram of the biochip testing system according to the second embodiment of the invention, where the biochip 7 has been carried on the recording medium 3;

FIG. 3 is a block diagram of the biochip testing system according to the third embodiment of the invention, where the biochip 7 has been carried on the recording medium 3;

FIG. 4 is an example where the biochip has adhered on a disc;

FIG. 5 is a personal digital biochip assistant according to the first embodiment of the invention;

FIG. 6 is a personal digital biochip assistant according to the second embodiment of the invent ion; and FIG. 7 is a perspective view of the cells of the biochip of the biochip testing system of the invention.

### DETAILED DESCRIPTION OF THE PREFERRED EMBODIMENTS OF THE INVENTION

A preferred embodiment of the invention will now be described hereinafter with reference to the drawings, where the same reference numerals indicate the same or similar elements.

The formation of the biochip is explained first by referring to FIG. 7. Generally, the formation of biochip 7 is to coat biological reagents on one end of a carrier 72 such as glass carrier, nylon carrier, or optical fiber carrier. The biological reagents include nucleic acids (DNA, RNA), proteins, peptides, saccharides, and the derivatives thereof. The end of carrier 72 being coated with biological reagents is then surrounded by an opaque clading 71.

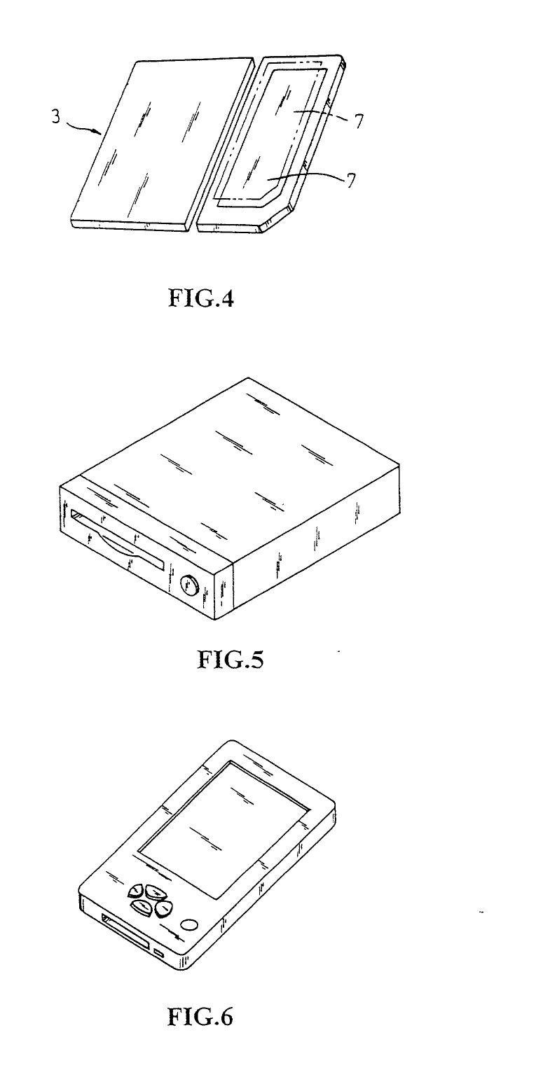

The biochip 7 is then detachably adhered to a recording medium 3, such as a disc (FIG. 4).

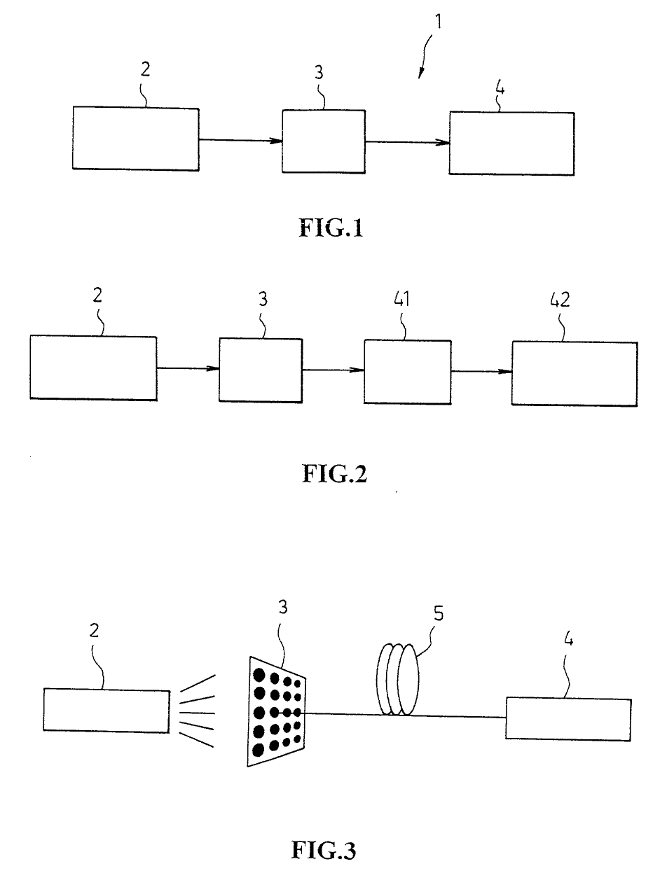

Subsequently, an explanation is made by referring to FIG. 1 where a block diagram of the biochip testing system according to the first embodiment of the invention is shown. In the first embodiment, the biochip 7 used herein is a transparent biochip. In FIG. 1, reference numeral 2 indicates a light transmitter, 3 indicates a recording medium carrying a biochip 7 thereon, and 4 indicates a light receiver. In the case that the light transmitter 2 is a light source, a laser light source, a LED light source, a LD light source, a haloid light source, an UV light source or other light sources can be used. By the construction shown in FIG. 1, after the light from light transmitter 2 passes through each cells of the biochip 7 and physically or chemically reacts with the biological reagents on each cells of the biochip 7, the light receiver 4 may receives a light passing through the cells coated with the biological reagents. By analyzing the variation of the light before and after the light passing through each cells of the biochip 7, a testing result of the cells of the biochip 7 can be obtained.

In the first embodiment of the invention, because the light transmitter 2 can directly emit light to the biochip 7, a testing result of the biochip 7 can be obtained through analyzing the light received on the light receiver 4. Therefore, the biochip testing system of the invention not only can radiate a light having high power without attenuation, but also has high resolution and low cost, which thus is suitable for use as personalized biochip testing system.

In the case that the biochip 7 is non-transparent, the light transmitter 2 and the light receiver 4 may be arranged on the same side with respect to the biochip 7, so as to obtain the variation of light through light reflection.

FIG. 2 shows the block diagram of the biochip testing system according to the second embodiment of the invention. In FIG. 2, the light receiver 4 includes a photoelectric converter 41 and a signal-processing unit 42. In the second embodiment, when an electronic signal is applied, the electronic signal may be converted as a light signal by an electro-optical converter (not shown in figures), so as to convert the electronic signal as a light signal to pass through the biochip 7. On the light receiver 4 side, the light signal being received is converted as electronic signal by the photoelectric converter 41, and then the electronic signal is processed with a voltage/current transform or analogue/digital transform by the signal processing unit 42 so as to compare with the original electronic signal. Through the comparison, the testing result of the biochip can be obtained.

In the second embodiment of the invention, because the light receiver 4 further includes a photoelectric converter 41 and a signal processing unit 42, what can be used as a signal source is not limited to a light source, and an electronic signal can also be used.

FIG. 3 shows the block diagram of the biochip testing system according to the third embodiment of the invention. Referring to FIG. 3, the differences between the third embodiment and the first embodiment is that the biochip 7 used in the third embodiment is an optical fiber biochip. The optical fiber biochip is formed by gathering a plurality of optical fibers and coating a biological reagent on one end of each optical fiber.

In the third embodiment, because the light signal is transmitted through the optical fiber 5, the power of the light signal being transmitted not only does not have attenuation, but also the resolution thereof is higher. Further, owing to the use of optical fiber biochip, the sizes of all testing points are the same and no interference will occur between the testing points.

In addition, in the embodiments of the invention, the biochip 7 is detachably adhered on the recording medium 3, where the recording medium 3 can be a disc as shown in FIG. 4. The other type of recording medium, such as an optical disc, smart card, etc., can also be used as the recording medium 3.

Furthermore, in the biochip testing system 1 of the invention, the light receiver 4 is arranged in a single one or in matrix, for one-by-one or in array sensoring each testing points where the signal is transmitted from the light transmitter 2 passing through or reflect from each cell of the biochip 7. In case that the light receiver 4 is a single one, it is used to scan each cell. Under this case, the cost is reduced while the scanning time is increased. However, if a matrix is taken, the scan time can be shortened.

In addition, in the biochip testing system 1 of the invention, the light receiver 4 may further comprise a writing head (not shown in figures), so as to write the signal on the recording medium 3. In other words, the testing result may be recorded on the recording medium 3.

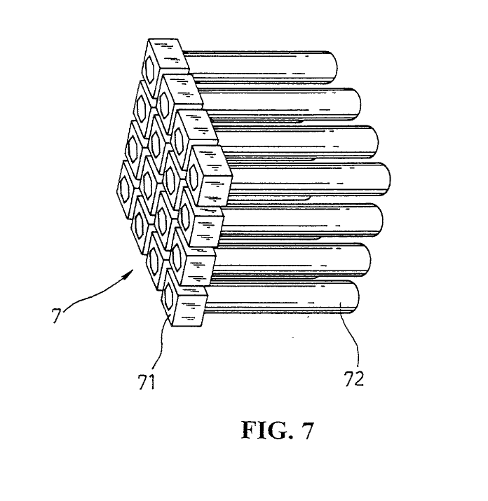

On the other hand, the invention also relates to a personal digital biochip assistant (hereinafter referred to PDBA) comprising the biochip testing system 1 as constructed above, as shown in FIGS. 5 and 6.

In the first embodiment of PDBA, as shown in FIG. 5, the PDBA is presented in a form of optical disc player. That is, the PDBA in optical disc player type comprises all elements of the biochip testing system 1 including the light transmitter 2, a biochip 7 coated with a biological reagents, a recording medium 3 for carrying the biochip 7, and a light receiver 4 for receiving the light from the light transmitter passing through or reflect from the biochip 7, where the biochip testing system 1 may be connected to an electronic devices such as a personal computer or a notebook, via an interface such as IEEE 1394 or universal serial bus (USB), or in wireless, so as to display the obtained analysis result in the display of the electronic devices.

In the second embodiment of PDBA, as shown in FIG. 6, the PDBA is presented in the form of a personal digital assistant (PDA). That is, the PDBA in personal digital assistant type comprises all elements of the biochip testing system 1 including the light transmitter 2, a biochip 7 coated with a biological reagents, a recording medium 3 for carrying the biochip 7, a light receiver 4 for receiving the light from the light transmitter passing through or reflect from the biochip 7, and an interface, such as IEEE 1394 or USB, where the biochip testing system 1 is integrally formed with an existing personal digital assistant.

By using the biochip testing system 1 of the invention, the gene data or the medical history of an individual can be recorded on the recording medium 3 having a biochip 7 thereon. Further, by using the PDBA of the invention shown in FIGS. 5 and 6, reading and writing of personal information from or on the recording medium 3 can be achieved with very low cost, and thus the invention may make the well-known expensive biochip testing system popular and commercialized.

The present disclosure includes that contained in the appended claims as well as that of the foregoing description. Although this invention has been described in its preferred forms with a certain degree of particularity, it is understood that the present disclosure of the preferred forms have been made only by way of example and numerous changes in the details of the construction and combination of arrangement of parts may be resorted to without departing from the spirit and scope of this invention.

What is claimed is:

1. A biochip testing system, comprising:
a light transmitter;
a biochip having a plurality of cells each coated with a biological reagent;
a recording medium for carrying the biochip; and
a light receiver for receiving the light from the light transmitter acting with the biological reagent on the biochip, thereby the status of each cell of the biochip may be tested through the variations of the light before and after passing the biochip.

2. The biochip testing system of claim 1, wherein the biochip is transparent.

3. The biochip testing system of claim 1, wherein the biochip is non-transparent.

4. The biochip testing system of claim 1, wherein the light transmitter includes a light source.

5. The biochip testing system of claim 4, wherein the light source is a laser light source, a LED light source, a LD light source, a haloid light source, or an UV light.

6. The biochip testing system of claim 1, wherein the biochip is formed polymer material carrier, such as glass carrier, nylon carrier, or optical fiber carrier, with one end of each carrier being coated with biological reagents thereon and surrounded by an opaque cladding. .

7. The biochip testing system of claim 6, wherein the optical fiber carrier is formed by gathering plural of plastic fibers, glass fibers, or quartz fibers.

8. The biochip testing system of claim 1, wherein the light receiver includes a photoelectric converter and a signal processing unit, where the photoelectric converter converted the light being received as an electronic signal, and the signal processing unit converted the electronic signal through current/voltage transform and analogue/digital transform as a digital signal and displayed on a monitor.

9. The biochip testing system of claim 1, where the light receiver is the one selecting from the group consisting of CMOS sensors, CCD array, CCDs, photodiode array, photodiodes, and PMT.

10. The biochip testing system of claim 8, wherein the electric signal may present on display of electronic devices, such as personal computer and notebook, or portable electronic devices, such as personal digital assistant (PDA), palm-size PC, or smartphone, through interface, such as IEEE 1394, USB included therein.

11. The biochip testing system of claim 8, wherein the electric signal may present on display of electronic devices, such as personal computer and notebook, or portable electronic devices, such as personal digital assistant (PDA), palm-size PC, or smartphone, in wireless.

12. The biochip testing system of claim 1, wherein the biochip is detachably adhered on the recording medium.

13. The biochip testing system of claim 1, wherein the light receiver is arranged in a single one or in matrix, so as to sensor the variation of light from the light source passing through or reflect from each cell of the biochip one-by-one or in array.

14. The biochip testing system of claim 1, wherein the light receiver further comprises a writing head for writing the variation sensored by the light receiver on the recording medium.

15. The biochip testing system of claim 1, wherein the recording medium is one selecting from the group consisting of magnetic disc, optical disc, or smart card.

16. A personal digital biochip assistant (PDBA), comprising the biochip testing system of any one of claims 1 to 15.

17. The personal digital biochip assistant of claim 16, wherein the PDBA presents its result in other electronic devices.

18. The personal digital biochip assistant of claim 16, wherein the PDBA is made all-in-one including display.
